(19) United States
(12) Patent Application Publication
(10) Pub. No.: US 2017/0333808 A1
(43) Pub. Date: Nov. 23, 2017

(54) DISTILLATION DEVICE

(71) Applicant: LG CHEM, LTD., Seoul (KR)

(72) Inventors: Sung Kyu LEE, Daejeon (KR); Joon Ho SHIN, Daejeon (KR)

(21) Appl. No.: 15/518,397

(22) PCT Filed: Nov. 2, 2015
(86) PCT No.: PCT/KR2015/011651

§ 371 Date: Apr. 11, 2017

(30) Foreign Application Priority Data
Oct. 31, 2014 (KR) 10-2014-0150416
Nov. 2, 2015 (KR) 10-2015-0153090

(51) Int. Cl. B01D 3/14; C07C 47/02; B01D 5/00

(52) U.S. Cl. CPC B01D 3/14; C07C 47/02; B01D 5/006; F25J 2200/80

(57) ABSTRACT

The present application relates to a distillation device. By using the distillation device of the present application, energy loss occurring during a purification process of a mixture including an isomer, for example, a raw material including n-butyl aldehyde and iso-butyl aldehyde, can be minimized, and a high-purity product can be separated, thus increasing economic efficiency of a process.

This application is a National Stage application of International Application No. PCT/KR2015/011651, filed Nov. 2, 2015, and claims the benefit of Korean Patent Application No. 10-2014-0150416, filed Oct. 31, 2014, and Korean Patent Application No. 10-2015-0153090, filed Nov. 2, 2015, and the contents of which are incorporated herein by reference in their entirety for all purposes as if fully set forth below.

### TECHNICAL FIELD

The present application relates to a distillation device for an isomer separation.

### BACKGROUND ART

Alkanols such as n-butanol are used in various applications such as, for example, a solvent for preparing a coating solution, in the chemical industry.

For example, n-butanol may be prepared through hydrogenation of n-butyl aldehyde. For example, butyl aldehyde may be prepared by allowing a gas mixture including propylene, carbon monoxide (CO), and hydrogen (H₂) to be subjected to oxo-reaction. In general, the prepared butyl aldehyde is a mixture of n-butyl aldehyde and iso-butyl aldehyde. N-butanol can be prepared by separating the n-butyl aldehyde from the mixture and allowing the separated n-butyl aldehyde to be subjected to hydrogenation.

In general cases, since a compound and an isomer thereof have a relatively small boiling point difference therebetween, compared to other compounds, it is difficult to separate the compound from the isomer thereof. For example, since a boiling point difference between n-butyl aldehyde and an isomer thereof, iso-butyl aldehyde, is very small, high energy is required to separate n-butyl aldehyde from iso-butyl aldehyde. Accordingly, considerable energy is consumed in obtaining high-purity n-butyl aldehyde and the purities of products may be decreased to reduce energy consumption in a separation process of the isomer.

### DISCLOSURE

### Technical Problem

Therefore, the present invention has been made in view of the above problems, and it is an object of the present application to provide a distillation device for high purity isomer separation with energy efficient.

### Technical Solution

The present application relates to a distillation device. In accordance with exemplary embodiments of the present application, the distillation device may increase separation efficiency of a process by minimizing energy loss occurring in a purification process of a mixture including an isomer, for example, a raw material including a compound represented by Formula 1 below and an isomer of the compound, and separation of high purity product. In particular, the distillation device of the present application provides optimized temperature and pressure for separation between n-butyl aldehyde and iso-butyl aldehyde using two distillation units, thus economically preparing high-purity n-butyl aldehyde.

Hereinafter, the distillation device of the present application will be described with reference to the accompanying drawings. The drawings are, however, provided as exemplary embodiments and the distillation device should not be understood as limited to the accompanying drawings.

FIG. 1 exemplarily illustrates a distillation device according to an embodiment of the present application. As illustrated in FIG. 1, in an exemplary embodiment, the distillation device includes two distillation units 10 and 20 and a heat exchanger 30. For example, the distillation device includes the first and second distillation units 10 and 20 and the heat exchanger 30. The first distillation unit 10 includes a first distillation column 100, a first condenser 101, a storage tank 102, and a first reboiler 103. The second distillation unit 20 includes a second distillation column 200, a second condenser 201, a storage tank 202, and a second reboiler 203.

The first and second distillation columns 100 and 200 are devices for various components separation included in a raw material using boiling point differences thereamong. In the distillation device of the present application, distillation columns with various shapes may be used, considering components of an introduced raw material or the boiling points of the components to be separated. A distillation column type which may be used in the distillation device of the present application is not specifically limited and may be, for example, a distillation column with a general structure as illustrated in FIG. 1 or a dividing wall-type distillation column including a dividing wall therein. In an example, the interiors of the first and second distillation columns 100 and 200 may be divided into upper sections 110 and 210, lower sections 130 and 230, and intermediate sections 120 and 220, as illustrated in FIG. 1. The expression ^{┌}upper section_{┘}, as used in the present specification, means relatively upper portions of the first and second distillation columns 100 and 200. For example, when each of the first and second distillation columns 100 and 200 is tri-sectioned in a height or length direction thereof, the upper section may mean an uppermost section thereof. In addition, the expression ^{┌}lower section_{┘} means a relatively lower portion of each of the first and second distillation columns 100 and 200. For example, when each of the first and second distillation columns 100 and 200 is tri-sectioned in a height or length direction thereof, the lower section may mean a lowest section thereof. In addition, when each of the first and second distillation columns 100 and 200 is tri-sectioned in a height or length direction thereof, the expression ^{┌}intermediate section_{┘} as used in the present specification may mean an intermediate section among three divided sections and a section between the upper section 110 or 210 and the lower section 130 or 230 of each of the first and second distillation columns 100 and 200. In the present specification, the upper section, the lower section and the intermediate section of the distillation column are relative concepts. The tops of the first and second distillation columns 100 and 200 are included in the upper sections and the bottoms of the first and second distillation columns 100 and 200 are included in the lower sections thereof. Unless mentioned otherwise, the upper section has the same meaning as “the top of a column”, and the lower section has the same meaning as “the bottom of a column”. The first and second distillation columns 100 and 200 may be distillation columns with a theoretical plate number of 50 to 150, 70 to 140, or 90 to 130. The expression ^{┌}theoretical plate number_{┘} means the number of hypothetical areas or plates, in which two phases such as a vapor phase and a liquid phase establish an equilibrium with each other, of the first and second distillation columns 100 and 200.

In an embodiment, as illustrated in FIG. 1, the first distillation unit 10 includes the first distillation column 100 and the first condenser 101, the storage tank 102, and the first reboiler 103 respectively connected to the first distillation column 100. The second distillation unit 20 includes the second distillation column 200 and the second condenser 201, the storage tank 202, and the second reboiler 203 respectively connected to the second distillation column 200, as illustrated in FIG. 1. For example, the first distillation column 100, the first condenser 101, the storage tank 102, and the first reboiler 103 may be fluidically connected to each other such that fluid introduced into the first distillation column 100 flows thereinto. The second distillation column 200, the second condenser 201, the storage tank 202, and the second reboiler 203 may be fluidically connected to each other such that fluid introduced into the second distillation column 200 flows thereinto. The ^{┌}condenser_{┘} is separately installed on the outside of the distillation column and performs cooling using, for example, a method of bringing a stream discharged from the top of the distillation column into contact with cooling water introduced from the outside. For example, the first condenser 101 of the first distillation column 100 may condense a first column top stream F₁₋₂ discharged from the upper section 110 of the first distillation column 100, and the second condenser 201 of the second distillation column 200 may condense a second column top stream F₂₋₂ discharged from the upper section 210 of the second distillation column 200. In addition, the ^{┌}reboiler_{┘} means a heating device separately installed on the outside of the distillation column. Alternatively, the reboiler may be a device for re-heating and evaporating a stream including components with a high boiling point discharged from the bottom of the distillation column. For example, the first reboiler 103 of the first distillation column 100 may be a device for heating a column bottom stream F₁₋₃ discharged from the lower section 130 of the first distillation column 100, and the second reboiler 203 of the second distillation column 200 described below may be a device for heating a column bottom stream F₂₋₃ discharged from the lower section 230 of the second distillation column 200. The ^{┌}storage tank_{┘} means a tank or a bath for temporarily storing a stream discharged from the distillation column and may be any tank or bath known in the art. For example, the first column top stream F₁₋₂ discharged from the upper section 110 of the first distillation column 100 is condensed in the first condenser 101, followed by being introduced into the storage tank 102 and stored therein. The second column top stream F₂₋₂ discharged from the upper section 210 of the second distillation column 200 may be condensed in the second condenser 201, followed by being introduced into the storage tank 202 and stored therein.

The first distillation column 100 includes a first supply port 121, and the second distillation column 200 includes a second supply port 221. In an embodiment, the first supply port 121 is located at the intermediate section 120 of the first distillation column 100, and the second supply port 221 is located at the intermediate section 220 of the second distillation column 200.

As illustrated in FIG. 1, the raw material including the compound represented by Formula 1 below and the isomer of the compound is introduced into the first supply port 121 of the first distillation column 100 and/or the second supply port 221 of the second distillation column 200:

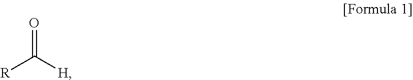

wherein R is a C₁ to C₁₂ alkyl group, for example, a C₁ to C₁₀ alkyl group, a C₁ to C₈ alkyl group, a C₁ to C₆ alkyl group, or a C₁ to C₄ alkyl group. In an example, the compound represented by Formula 1 may be, for example, n-butyl aldehyde, and the isomer of the compound may be iso-butyl aldehyde.

For example, as illustrated in FIG. 1, in the case of the distillation device with a structure wherein the first distillation column 100 and the second distillation column 200 are connected in parallel (hereinafter also referred to as a ^{┌}parallel structure_{┘}), the raw material including the compound represented by Formula 1 and the isomer of the compound is introduced into the first supply port 121 of the first distillation column 100 and the second supply port 221 of the second distillation column 200, respectively. When the distillation device of the present application includes the first distillation column 100 and the second distillation column 200 which are connected to each other in parallel, as illustrated in FIG. 1, energy reduction effects may be maximized.

In an example, the raw material introduced into the first supply port 121 of the first distillation column 100 is introduced into the intermediate section 120 of the first distillation column 100, and a raw material F₁₋₁ introduced into the intermediate section 120 of the first distillation column 100 is separately discharged into each of a column top stream discharged from the upper section 110 of the first distillation column 100 and a column bottom stream discharged from the lower section 130 of the first distillation column 100. In this case, the column bottom stream discharged from the lower section 130 of the first distillation column 100 may be separately discharged into at least one stream. For example, the raw material introduced into the first distillation column 100 may be separately discharged into each of the first column top stream F₁₋₂, and first, second and third column bottom streams F₁₋₃, F₁₋₄, and F₁₋₅ discharged from the lower section 130 of the first distillation column 100. The first column top stream F₁₋₂ discharged from the upper section 110 of the first distillation column 100 is introduced into the first condenser 101, a portion or all of the first column top stream F₁₋₂ passing through the first condenser 101 may be refluxed in the upper section 110 of the first distillation column 100 or stored as a product. In an example, a stream discharged from the first condenser 101 may be introduced into the storage tank 102 and stored therein, followed by being refluxed in the first distillation column 100 or stored as a product. In addition, the first column bottom stream F₁₋₃ discharged from the lower section 130 of the first distillation column 100 is introduced into the first reboiler 103. The first column bottom stream F₁₋₃ passing through the first reboiler 103 is introduced into the lower section 130 of the first distillation column 100 and the second column bottom stream F₁₋₄ discharged from the lower section 130 of the first distillation column 100 may be stored as a product. The first column bottom stream F₁₋₃ introduced into the first reboiler 103 may be heated by high-pressure steam passing through the first reboiler 103. The amount of this high-pressure steam may be properly controlled by the heat exchanger 30 described below. For example, when heat exchange in the heat exchanger 30 is sufficiently carried out, the high-pressure steam might not be used at all. However, when heat exchange is not smoothly carried out due to disturbance of a discharge rate of a raw material or a process, separation efficiency may be rapidly decreased. Accordingly, a proper amount of the high-pressure steam may be temporarily used such that robust separation efficiency can be maintained despite the disturbance.

As described above, in the case of a distillation device including the first distillation column 100 and the second distillation column 200 connected in parallel, a stream introduced into the second supply port 221 of the second distillation column 200 may include the raw material including the compound represented by Formula 1 and the isomer of the compound. The raw material introduced into the second supply port 221 of the second distillation column 200 is introduced into the intermediate section 220 of the second distillation column 200. A raw material F₂₋₁ introduced into the intermediate section 220 of the second distillation column 200 is separately discharged into a column top stream discharged from the upper section 210 of the second distillation column 200 and a column bottom stream discharged from the lower section 230 of the second distillation column 200. In this case, the column bottom stream discharged from the lower section 230 of the second distillation column 200 may be separately discharged into at least one stream. For example, the raw material introduced into the second distillation column 200 may be separately discharged into each of the second column top stream F₂₋₂ and a fourth column bottom stream F₂₋₃ and a fifth column bottom stream F₂₋₄ discharged from the lower section 230 of the second distillation column 200. The fourth column bottom stream F₂₋₃ discharged from the lower section 230 of the second distillation column 200 is introduced into the second reboiler 203. The fourth column bottom stream F₂₋₃ passing through the second reboiler 203 is introduced into the lower section 230 of the second distillation column 200, and the fifth column bottom stream F₂₋₄ discharged from the lower section 230 of the second distillation column 200 may be stored as a product.

The third column bottom stream F₁₋₅ discharged from the lower section 130 of the first distillation column 100 and the second column top stream F₂₋₂ discharged from the upper section 210 of the second distillation column 200 are introduced into the heat exchanger 30. The ^{┌}heat exchanger_{┘} is separately installed on the outside of the distillation column and performs heat exchange such that heat transfer between two fluid streams, the temperatures of which are different, is fluently carried out. For example, the heat exchanger 30 may allow heat exchange between the third column bottom stream F₁₋₅ discharged from the lower section 130 of the first distillation column 100 and the second column top stream F₂₋₂ discharged from the upper section 210 of the second distillation column 200. In the distillation device of the present application, the third column bottom stream F₁₋₅ with a high boiling point discharged from the lower section 130 of the first distillation column 100 and the second column top stream F₂₋₂ with a low boiling point discharged from the upper section 210 of the second distillation column 200 exchange heat in the heat exchanger 30, thereby reducing energy required in condensation and heating processes in which the condenser or the reboiler is used.

The heat exchanger 30 may be directly or indirectly connected to pipes through which the third column bottom stream F₁₋₅ of the first distillation column 100 and the second column top stream F₂₋₂ of the second distillation column 200 pass. In an example, when the heat exchanger 30 is directly connected to the pipes through which the third column bottom stream F₁₋₅ of the first distillation column 100 and the second column top stream F₂₋₂ of the second distillation column 200 pass, heat exchange between the third column bottom stream F₁₋₅ and the second column top stream F₂₋₂ may be efficiently performed.

Heat exchange between the third column bottom stream F₁₋₅ and the second column top stream F₂₋₂ introduced into the heat exchanger 30 is carried out, the third column bottom stream F₁₋₅ passing through the heat exchanger 30 is refluxed in the lower section 130 of the first distillation column 100, the second column top stream F₂₋₂ passing through the heat exchanger 30 is introduced into the second condenser 201, and a portion or all of the second column top stream F₂₋₂ passing through the second condenser 201 may be refluxed in the upper section 210 of the second distillation column 200 or stored as a product. In an example, a stream discharged from the second condenser 201 is introduced in the storage tank 202 and stored therein. Subsequently, the stored stream may be refluxed in the second distillation column 200 or stored as a product.

In the heat exchanger 30, the third column bottom stream F₁₋₅ may be heat exchanged with the second column top stream F₂₋₂ before the third column bottom stream F₁₋₅ is refluxed in the first distillation column 100, and the second column top stream F₂₋₂ may exchange heat with the third column bottom stream F₁₋₅ before the second column top stream F₂₋₂ is introduced into the second condenser 201. For example, the second column top stream F₂₋₂ including a low boiling component discharged from the upper section 210 of the second distillation column 200 stops by the heat exchanger 30 before being refluxed in the upper section 210 of the second distillation column 200. At this time, heat is supplied to the heat exchanger 30. Accordingly, the second column top stream F₂₋₂ discharged from the second distillation column 200 may be refluxed, at a relatively low temperature, into the second distillation column 200. Accordingly, the quantity of heat needed for condensing the second column top stream F₂₋₂ discharged from the upper section 210 of the second distillation column 200 may be decreased, and costs necessary for the condensation process may be reduced by decreasing the amount of cooling water used in the condensation process in which the second condenser 201 is used. In addition, the third column bottom stream F₁₋₅, as a stream including a high boiling component discharged from the lower section 130 of the first distillation column 100 drops by the heat exchanger 30 before being refluxed in the lower section 130 of the first distillation column 100. At this time, heat transferred by the second column top stream F₂₋₂ may be supplied to the third column bottom stream F₁₋₅. Accordingly, the second column top stream F₂₋₂ supplies heat to the lower section 130 of the first distillation column 100 and thus the amount of steam used in the first reboiler 103 in order to heat the first column bottom stream F₁₋₃ discharged from the lower section 130 of the first distillation column 100 is reduced, thereby reducing costs.

Hereinafter, a separation process between n-butyl aldehyde and iso-butyl aldehyde, as an isomer thereof, using a distillation device according to an embodiment of the present application will be described in detail.

In an example, the raw material F₁₋₁ including n-butyl aldehyde and iso-butyl aldehyde, as an isomer thereof, is introduced into each of the first supply port 121 of the first distillation column 100 and the second supply port 221 of the second distillation column 200.

In this case, a stream including a large amount of iso-butyl aldehyde with a relatively low boiling point among components that are included in the raw material F₁₋₁ introduced into the first supply port 121 may be discharged as the first column top stream F₁₋₂ from the upper section 110 of the first distillation column 100, and a stream including a large amount of n-butyl aldehyde with a relatively high boiling point may be discharged as the first, second and third column bottom streams F₁₋₃, F₁₋₄, and F₁₋₅ from the lower section 130 of the first distillation column 100. The first column top stream F₁₋₂ discharged from the upper section 110 of the first distillation column 100 is introduced into the storage tank 102 via the first condenser 101. A portion of the stream discharged from the storage tank 102 is refluxed in the upper section 110 of the first distillation column 100, and a portion of the remainder of the stream may be stored as a product. The product may be high-purity iso-butyl aldehyde. Meanwhile, the first column bottom stream F₁₋₃ discharged from the lower section 130 of the first distillation column 100 may be refluxed in the lower section 130 of the first distillation column 100 via the first reboiler 103, and the second column bottom stream F₁₋₄ may be stored as a product. The product may be high-purity n-butyl aldehyde. In addition, the third column bottom stream F₁₋₅ may exchange heat with the second column top stream F₂₋₂ of the second distillation column 200 in the heat exchanger 30, followed by being refluxed in the lower section 130 of the first distillation column 100.

In addition, a stream including a large amount of iso-butyl aldehyde with a relatively low boiling point among components included in the raw material stream F₂₋₁ introduced into the second supply port 221 may be discharged as the second column top stream F₂₋₂ from the upper section 210 of the second distillation column 200, and a stream including a large amount of n-butyl aldehyde with a relatively high boiling point among the components may be discharged as the fourth and fifth column bottom streams F₂₋₃ and F₂₋₄ from the lower section 230 of the second distillation column 200. The discharged second column top stream F₂₋₂ exchanges heat with the third column bottom stream F₁₋₅ of the first distillation column 100 in the heat exchanger 30 and is then introduced into the storage tank 202 via the second condenser 201. A portion of the stream discharged from the storage tank 202 may be refluxed in the upper section 210 of the second distillation column 200, and another portion of the remainder of the stream may be stored as a product. The product may be high-purity iso-butyl aldehyde. In addition, a stream including a component with a relatively high boiling point among components included in the raw material F₂₋₁ may be discharged as the fourth and fifth column bottom streams F₂₋₃ and F₂₋₄ from the lower section 230 of the second distillation column 200. The fourth column bottom stream F₂₋₃ may be refluxed in the lower section 230 of the second distillation column 200 via the second reboiler 203 and the fifth column bottom stream F₂₋₄ may be stored as a product. The product may be high-purity n-butyl aldehyde.

In the present specification, the expression ^{┌}stream including a low boiling component_{┘} means a stream including a large amount of a component with a relatively low boiling point in a raw material stream including a low boiling component and a high boiling component. For example, the stream including a low boiling component means streams discharged from the upper sections 110 and 210 of the first and second distillation columns 100 and 200. In addition, the expression ^{┌}stream including a high boiling component_{┘} means a stream including a large amount of a component with a relatively high boiling point in a raw material stream including a low boiling component and a high boiling component. For example, the stream including a high boiling component means a stream including a large amount of a component with a relatively high boiling point discharged from the lower sections 130 and 230 of the first and second distillation columns 100 and 200. The expression ^{┌}stream including a large amount of a component_{┘} means a stream wherein the content of each of a low boiling component, which is included in a stream discharged from the upper sections 110 and 210 of the first and second distillation columns 100 and 200, and a high boiling component, which is included in stream discharged from the lower sections 130 and 230 of the first and second distillation columns 100 and 200, is higher than the content of each of the low boiling component and the high boiling component included in the raw material F₁₋₁. For example, the content of each of the low boiling component included in the first column top stream F₁₋₂ of the first distillation column 100 and the low boiling component included in the second column top stream F₂₋₂ of the second distillation column 200 may be 50% by weight or more, 80% by weight or more, 90% by weight or more, 95% by weight or more or 99% by weight or more. Alternatively, the contents of a high boiling component included in each of the first column top stream F₁₋₂, and the first, second and third column bottom streams F₁₋₃, F₁₋₄, and F₁₋₅ of the first distillation column 100 and a high boiling component included in each of the fourth and fifth column bottom streams F₂₋₃ and F₂₋₄ of the second distillation column 200 may respectively be 50% by weight or more, 80% by weight or more, 90% by weight or more, 95% by weight or more, or 99% by weight or more.

FIG. 2 exemplarily illustrates a distillation device according to another embodiment of the present application.

As illustrated in FIG. 2, when a distillation device includes the first distillation column 100 and the second distillation column 200 connected in series (hereinafter also referred to as a ^{┌}serial structure_{┘}), the raw material including the compound represented by Formula 1 and the isomer of the compound is introduced into the first supply port 121 of the first distillation column 100. In this case, the second column bottom stream F₁₋₄ of the first distillation column 100 is introduced into the second supply port 221 of the second distillation column 200. As illustrated in FIG. 2, when the distillation device of the present application includes the first distillation column 100 and the second distillation column 200 connected in series, the purity of prepared n-butyl aldehyde may be maximized.

In an example, as illustrated in FIG. 2, the raw material introduced into the first supply port 121 of the first distillation column 100 is introduced into the intermediate section 120 of the first distillation column 100, and the raw material F₁₋₁ introduced into the intermediate section 120 of the first distillation column 100 is separately discharged into each of a column top stream discharged from the upper section 110 of the first distillation column 100 and a column bottom stream discharged from the lower section 130 of the first distillation column 100. In this case, as in the aforementioned distillation device with a parallel structure, the column bottom stream discharged from the lower section 130 of the first distillation column 100 may be separately discharged into at least one stream. For example, the raw material introduced into the first distillation column 100 may be separately discharged into each of the first column top stream F₁₋₂, and the first, second and third column bottom streams F₁₋₃, F₁₋₄, and F₁₋₅ discharged from the lower section 130 of the first distillation column 100. The first column top stream F₁₋₂ discharged from the upper section 110 of the first distillation column 100 is introduced into the first condenser 101, a portion or all of the first column top stream F₁₋₂ passing through the first condenser 101 is refluxed in the upper section 110 of the first distillation column 100 or may be stored as a product. In an example, a stream discharged from the first condenser 101 may be introduced into the storage tank 102 and stored therein, followed by being refluxed in the first distillation column 100 or stored as a product. In addition, the first column bottom stream F₁₋₃ discharged from the lower section 130 of the first distillation column 100 is introduced into the first reboiler 103. The first column bottom stream F₁₋₃ passing through the first reboiler 103 may be introduced into the lower section 130 of the first distillation column 100.

As described above, in the case of a distillation device including the first distillation column 100 and the second distillation column 200 connected in series, a stream introduced into the second supply port 221 of the second distillation column 200 may be the second column bottom stream F₁₋₄ of the first distillation column 100. The second column bottom stream F₁₋₄ introduced into the second supply port 221 of the second distillation column 200 is introduced into the intermediate section 220 of the second distillation column 200. The second column bottom stream F₁₋₄ introduced into the intermediate section 220 of the second distillation column 200 is separately discharged into each of a column top stream discharged from the upper section 210 of the second distillation column 200 and a column bottom stream discharged from the lower section 230 of the second distillation column 200. In this case, as in the aforementioned distillation device with a parallel structure, the column bottom stream discharged from the lower section 230 of the second distillation column 200 may be separately discharged into at least one stream. For example, the stream introduced into the second distillation column 200 may be separately discharged into each of the second column top stream F₂₋₂ and the fourth column bottom stream F₂₋₃ and the fifth column bottom stream F₂₋₄ discharged from the lower section 230 of the second distillation column 200. The fourth column bottom stream F₂₋₃ discharged from the lower section 230 of the second distillation column 200 is introduced into the second reboiler 203. The fourth column bottom stream F₂₋₃ passing through the second reboiler 203 is introduced into the lower section 230 of the second distillation column 200, and the fifth column bottom stream F₂₋₄ discharged from the lower section 230 of the second distillation column 200 may be stored as a product.

The third column bottom stream F₁₋₅ discharged from the lower section 130 of the first distillation column 100 and the second column top stream F₂₋₂ discharged from the upper section 210 of the second distillation column 200 are introduced into the heat exchanger 30. As described above, the heat exchanger 30 may allow heat exchange between the third column bottom stream F₁₋₅ discharged from the lower section 130 of the first distillation column 100 and the second column top stream F₂₋₂ discharged from the upper section 210 of the second distillation column 200. In the distillation device of the present application, the third column bottom stream F₁₋₅ with a high boiling point discharged from the lower section 130 of the first distillation column 100 and the second column top stream F₂₋₂ with a low boiling point discharged from the upper section 210 of the second distillation column 200 are heat-exchanged between each other in the heat exchanger 30, thereby reducing energy required in condensation and heating processes, in which the condenser or the reboiler is used, and preparing high-purity n-butyl aldehyde.

The heat exchanger 30 is the same as that for the aforementioned distillation device including the first distillation column 100 and the second distillation column 200 which are connected to each other in parallel, and description thereof is thus omitted.

Hereinafter, a process of separating n-butyl aldehyde and iso-butyl aldehyde, as an isomer thereof, will be described in more detail using a distillation device including the first distillation column 100 and the second distillation column 200, which are connected in series, according to another embodiment of the present application.

In an example, the raw material F₁₋₁ including n-butyl aldehyde and iso-butyl aldehyde, as an isomer thereof is introduced into the first supply port 121 of the first distillation column 100.

In this case, a stream including a large amount of iso-butyl aldehyde with a relatively low boiling point among components that are included in the raw material F₁₋₁ introduced into the first supply port 121 may be discharged as the first column top stream F₁₋₂ from the upper section 110 of the first distillation column 100, and a stream including a large amount of n-butyl aldehyde with a relatively high boiling point may be discharged as the first, second and third column bottom streams F₁₋₃, F₁₋₄, and F₁₋₅ from the lower section 130 of the first distillation column 100. The first column top stream F₁₋₂ discharged from the upper section 110 of the first distillation column 100 is introduced into the storage tank 102 via the first condenser 101. A portion of a stream discharged from the storage tank 102 is refluxed in the upper section 110 of the first distillation column 100, and a portion of the remainder of the stream may be stored as a product. The product may be high purity iso-butyl aldehyde. Meanwhile, the first column bottom stream F₁₋₃ discharged from the lower section 130 of the first distillation column 100 may be refluxed in the lower section 130 of the first distillation column 100 via the first reboiler 103, and the second column bottom stream F₁₋₄ may be introduced into the second supply port 221 of the second distillation column 200. In addition, the third column bottom stream F₁₋₅ may exchange heat with the second column top stream F₂₋₂ of the second distillation column 200 in the heat exchanger 30, followed by being refluxed in the lower section 130 of the first distillation column 100.

In addition, the second column bottom stream F₁₋₄ introduced into the second supply port 221 includes n-butyl aldehyde and a high boiling component. Accordingly, a stream including a large amount of n-butyl aldehyde with a relatively low boiling point among components included in the second column bottom stream F₁₋₄ may be discharged as the second column top stream F₂₋₂ from the upper section 210 of the second distillation column 200, and a stream including components with relatively high boiling points may be discharged as the fourth and fifth column bottom streams F₂₋₃ and F₂₋₄ from the lower section 230 of the second distillation column 200. The discharged second column top stream F₂₋₂ exchanges heat with the third column bottom stream F₁₋₅ of the first distillation column 100 in the heat exchanger 30 and is then introduced into the storage tank 202 via the second condenser 201. A portion of the stream discharged from the storage tank 202 may be refluxed in the upper section 210 of the second distillation column 200, and a portion of the remainder of the stream may be stored as a product. The product may be ultrahigh-purity n-butyl aldehyde. In addition, a stream including a component with a relatively high boiling point among components included in the second column top stream F₂₋₂ may be discharged as the fourth and fifth column bottom streams F₂₋₃ and F₂₋₄ from the lower section 230 of the second distillation column 200. The fourth column bottom stream F₂₋₃ may be refluxed in the lower section 230 of the second distillation column 200 via the second reboiler 203 and the fifth column bottom stream F₂₋₄ may be stored as a product. The product may include, for example, n-butyl aldehyde, butyl alcohol, or dimers thereof, and trimers thereof.

In an example, a portion of the fifth column bottom stream F₂₋₄ discharged from the lower section 230 of the second distillation column 200 may be introduced into the lower section 130 of the first distillation column 100, for example, a 45th to 145th plate of the first distillation column 100 with a theoretical plate number of 50 to 150. Accordingly, n-butyl aldehyde that may remain in the fifth column bottom stream F₂₋₄ may be supplied to the lower section 130 of the first distillation column 100, thereby preparing n-butyl aldehyde with higher purity. In this case, a ratio of a discharge rate (ton/hr) of the stream introduced into the lower section 130 of the first distillation column 100 to a discharge rate (ton/hr) of the fifth column bottom stream F₂₋₄ discharged from the lower section 230 of the second distillation column 200 may be 1:0.85 to 1:0.95. By controlling the flow ratio of the stream introduced into the lower section 130 of the first distillation column 100 within this range, n-butyl aldehyde with higher purity may be prepared.

In an embodiment, the distillation device of the present application satisfies Equation 1 below.

Tₜ₋₂−T_{b-3}≧8° C. [Equation 1]

wherein Tₜ₋₂ indicates a temperature of the second column top stream F₂₋₂, and T_{b-3} indicates a temperature of the third column bottom stream F₁₋₅.

When the distillation device of the present application satisfies Equation 1, the compound represented by Formula 1, particularly n-butyl aldehyde, may be separated in superior efficiency and high purity using the distillation device with the aforementioned parallel structure or serial structure. That is, by controlling the distillation device such that a temperature difference between the second column top stream F₂₋₂ and the third column bottom stream F₁₋₅ satisfies Equation 1, heat exchange efficiency between the second column top stream F₂₋₂ and the third column bottom stream F₁₋₅ may be maximized. Accordingly, the compound represented by Formula 1, particularly n-butyl aldehyde, may be separated in superior efficiency and high purity.

In an example, so long as a temperature difference between the second column top stream F₂₋₂ discharged from the upper section 210 of the second distillation column 200 and the third column bottom stream F₁₋₅ discharged from the lower section 130 of the first distillation column 100 satisfies Equation 1, there is no specific limitation. For example, the temperature difference may be 8° C. or more, 9° C. or more, 10° C. or more, or 13° C. or more. Since heat exchange efficiency is superior with increasing temperature difference between the second column top stream F₂₋₂ discharged from the upper section 210 of the second distillation column 200 and the third column bottom stream F₁₋₅ discharged from the lower section 130 of the first distillation column 100, the maximum value of the temperature difference is not specifically limited. For example, a temperature difference between the second column top stream F₂₋₂ discharged from the upper section 210 of the second distillation column 200 and the third column bottom stream F₁₋₅ discharged from the lower section 130 of the first distillation column 100 may be 100° C. or less, considering process efficiency.

In an example, the distillation device of the present application satisfies Equation 2 below:

P₂/P₁≧20, [Equation 2]

wherein P₁ indicates a pressure (kg/cm²g) of the upper section 110 of the first distillation column 100, and P₂ indicates a pressure (kg/cm²g) of the upper section 210 of the second distillation column 200.

When the distillation device of the present application satisfies Equation 2, the compound represented by Formula 1, particularly n-butyl aldehyde, may be separated in superior efficiency and high purity using the distillation device with the aforementioned parallel structure or serial structure. That is, by controlling the distillation device such that a ratio of the pressure of the upper section 210 of the second distillation column 200 to the pressure of the upper section 110 of the first distillation column 100 satisfies Equation 2, heat exchange efficiency between the second column top stream F₂₋₂ and the third column bottom stream F₁₋₅ may be maximized. Accordingly, the compound represented by Formula 1, particularly n-butyl aldehyde, may be separated in superior efficiency and high purity.

For example, in order to increase the heat exchange efficiency of the heat exchanger 30, the interior temperature of the first distillation column 100 may be kept lower than the interior temperature of the second distillation column 200, and thus, the pressure of the upper section 110 of the first distillation column 100 may be kept lower than that of the upper section 210 of the second distillation column 200.

In an example, so long as a ratio of the pressure of the upper section 210 of the second distillation column 200 to the pressure of the upper section 110 of the first distillation column 100 satisfies Equation 2, there is no specific limitation. For example, the ratio may be 20 or more, 25 or more, 35 or more, 50 or more, 80 or more, or 120 or more. Since heat exchange efficiency improves with increasing ratio of the pressure of the upper section 210 of the second distillation column 200 to the pressure of the upper section 110 of the first distillation column 100, the maximum value of the ratio is not specifically limited. For example, the ratio of the pressure of the upper section 210 of the second distillation column 200 to the pressure of the upper section 110 of the first distillation column 100 may be 300 or less, or 200 or less, considering process efficiency.

When the distillation device of the present application has the aforementioned parallel structure, the temperature of the second column top stream F₂₋₂ discharged from the upper section 210 of the second distillation column 200 is not specifically limited so long as Equation 1 is satisfied. The temperature may be 100 to 110° C., for example, 102° C. to 108° C. or 104° C. to 106° C. In addition, the temperature of the third column bottom stream F₁₋₅ discharged from the lower section 130 of the first distillation column 100 is not specifically limited so long as Equation 1 is satisfied. The temperature may be 90° C. to 100° C., for example, 92° C. to 98° C. or 94° C. to 96° C. In this case, the pressure of the upper section 110 of the first distillation column 100 is not specifically limited so long as Equation 2 is satisfied. The pressure may be 0.01 to 0.1 Kg/cm²g, 0.01 to 0.07 Kg/cm²g, or 0.015 to 0.03 Kg/cm²g. In addition, the pressure of the upper section 210 of the second distillation column 200 is not specifically limited so long as Equation 2 is satisfied. The pressure may be 2.3 to 2.7 Kg/cm²g, 2.35 to 2.65 Kg/cm²g, or 2.4 to 2.6 Kg/cm²g.

In an example, when the distillation device of the present application has the aforementioned parallel structure, the temperature of the upper section 110 of the first distillation column 100 may be 60° C. to 70° C., for example, 62° C. to 68° C. or 64° C. to 66° C., and the temperature of the lower section 130 of the first distillation column 100 may be 90° C. to 100° C., for example, 92° C. to 98° C. or 94° C. to 96° C., but the present application is not limited thereto. In this case, the temperature of the upper section 210 of the second distillation column 200 may be 100 to 110° C., for example, 102° C. to 108° C. or 104° C. to 106° C., and the temperature of the lower section 230 of the second distillation column 200 may be 120 to 140° C., for example, 124° C. to 138° C. or 126° C. to 134 V, but the present application is not limited thereto.

In an example, when the distillation device of the present application has the aforementioned parallel structure, Equation 3 below may be satisfied.

0.3≦F₁/F₂≦3.0 [Equation 3]

wherein F₁ indicates a discharge rate of a raw material (ton/hr) introduced into the first supply port 121 of the first distillation column 100, and F₂ indicates a discharge rate of a raw material (ton/hr) introduced into the second supply port 221 of the second distillation column 200.

In the distillation device, energy reduction effect may be maximized by controlling a ratio of the discharge rate of the raw material F₁₋₁ introduced into the first supply port 121 of the first distillation column 100 to the discharge rate of the raw material F₂₋₁ introduced into the second supply port 221 of the second distillation column 200 within the range of Equation 3.

In an example, the ratio of the discharge rate of the raw material F₁₋₁ introduced into the first supply port 121 of the first distillation column 100 to the discharge rate of the raw material F₂₋₁ introduced into the second supply port 221 of the second distillation column 200 is not specifically limited so long as the ratio is within the aforementioned range. For example, the ratio may be 0.3 to 3.0, 0.6 to 2.0, 0.7 to 1.7, 0.8 to 1.4, or 0.9 to 1.2.

In addition, the discharge rate of the raw material F₁₋₁ introduced into the first supply port 121 of the first distillation column 100 is not specifically limited so long as Equation 3 is satisfied, and may be 10 to 30 ton/hr, for example, 14 to 26 ton/hr or 18 to 22 ton/hr. The discharge rate of the raw material F₂₋₁ introduced into the second supply port 221 of the second distillation column 200 is not specifically limited so long as Equation 3 is satisfied, and may be 10 to 30 ton/hr, for example, 14 to 26 ton/hr or 18 to 22 ton/hr.

When the distillation device of the present application has the aforementioned parallel structure, the content of iso-butyl aldehyde in each of the first column top stream F₁₋₂ discharged from the upper section 110 of the first distillation column 100 and the second column top stream F₂₋₂ discharged from the upper section 210 of the second distillation column 200 may be 90% or more, preferably 99% or more. The content of n-butyl aldehyde in each of the second column bottom stream F₁₋₄ discharged from the lower section 130 of the first distillation column 100 and the fifth column bottom stream F₂₋₄ discharged from the lower section 230 of the second distillation column 200 may be 90% or more, preferably 99% or more.

When the distillation device of the present application has the aforementioned serial structure, the temperature of the second column top stream F₂₋₂ discharged from the upper section 210 of the second distillation column 200 is not specifically limited so long as Equation 1 is satisfied, and may be 100 to 110° C., for example, 102° C. to 108° C. or 104° C. to 106° C. In addition, the temperature of the third column bottom stream F₁₋₅ discharged from the lower section 130 of the first distillation column 100 is not specifically limited so long as Equation 1 is satisfied, and may be 90° C. to 100° C., for example, 92° C. to 98° C. or 94° C. to 96° C. In this case, the pressure of the upper section 110 of the first distillation column 100 is not specifically limited so long as Equation 2 is satisfied, and may be 0.01 to 0.1 Kg/cm²g, 0.012 to 0.07 Kg/cm²g, or 0.015 to 0.03 Kg/cm²g. In addition, the pressure of the upper section 210 of the second distillation column 200 is not specifically limited so long as Equation 2 is satisfied, and may be 1.0 to 2.0 Kg/cm²g, 1.2 to 2.0 Kg/cm²g, or 1.4 to 1.6 Kg/cm²g.

In an example, when the distillation device of the present application has the aforementioned serial structure, the temperature of the upper section 110 of the first distillation column 100 may be 60° C. to 70° C., for example, 62° C. to 68° C. or 64° C. to 66° C., and the temperature of the lower section 130 of the first distillation column 100 may be 90° C. to 100° C., for example, 92° C. to 98° C. or 94° C. to 96° C., but the present application is not limited thereto. In this case, the temperature of the upper section 210 of the second distillation column 200 may be 100 to 110° C., for example, 102° C. to 108° C. or 104° C. to 106° C., and the temperature of the lower section 230 of the second distillation column 200 may be 120 to 140° C., for example, 124° C. to 138° C. or 126° C. to 134 V, but the present application is not limited thereto.

When the distillation device of the present application has the aforementioned serial structure, the content of iso-butyl aldehyde in the first column top stream F₁₋₂ discharged from the upper section 110 of the first distillation column 100 may be 90% or more, preferably 99% or more, and the content of n-butyl aldehyde in the second column top stream F₂₋₂ discharged from the upper section 210 of the second distillation column 200 may be 90% or more, preferably 99% or more.

The present application also relates to a method of preparing the compound represented by Formula 1.

The preparation method according to an exemplary embodiment of the present application may be carried out using the aforementioned distillation device, and thus, the same contents as the descriptions of the aforementioned distillation device are omitted.

In an embodiment, the preparation method of the present application includes i) a step of introducing the raw material including the compound represented by Formula 1 below and the isomer of the compound into each of the first supply port 121 of the first distillation column 100 and the second supply port 221 of the second distillation column 200; ii) a step of discharging the raw material introduced into the first supply port 121 to each of the first column top stream F₁₋₂ discharged from the upper section 110 of the first distillation column 100; and the first column top stream F₁₋₂, and the first, second and third column bottom streams F₁₋₃, F₁₋₄, and F₁₋₅ discharged from the lower section 130 of the first distillation column 100; iii) a step of discharging the raw material introduced into the second supply port 221 to each of the second column top stream F₂₋₂ discharged from the upper section 210 of the second distillation column 200; and the fourth and fifth column bottom streams F₂₋₃ and F₂₋₄ discharged from the lower section 230 of the second distillation column 200; iv) a step of exchanging heat between the second column top stream F₂₋₂ and the third column bottom stream F₁₋₅; and v) a step of separating the compound represented by Formula 1 from the lower section 130 of the first distillation column 100, and the isomer of the compound represented by Formula 1 from the upper section 110 of the first distillation column 100 and the upper section 210 of the second distillation column 200:

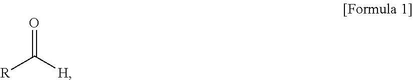

wherein R is a C₁ to C₁₂ alkyl group. The compound represented by Formula 1 may be, for example, n-butyl aldehyde or iso-butyl aldehyde. In an example, the compound represented by Formula 1 may be n-butyl aldehyde.

The preparation method may be carried out using a distillation device with the aforementioned parallel structure. The distillation device with the parallel structure is the same as those described above, description thereof thus being omitted.

Steps i) to v) are each independently, organically connected, and thus, boundaries therebetween are not clearly divided according to chronological order. Each of steps i) to v) may be carried out sequentially or independently at the same time.

The preparation method satisfies Equations 1 and 2 below. Descriptions therefor are the same as those described above, thus being omitted.

Tₜ₋₂−T_{b-3}≧8° C. [Equation 1]

P₂/P₁≧20 [Equation 2]

wherein Tₜ₋₂ indicates a temperature of the second column top stream F₂₋₂, and T_{b-3} indicates a temperature of the third column bottom stream F₁₋₅, and

P₁ indicates a pressure (kg/cm²g) of the upper section 110 of the first distillation column 100, and P₂ indicates a pressure (kg/cm²g) of the upper section 210 of the second distillation column 200.

In another embodiment, the preparation method of the present application includes a) a step of introducing the raw material including the compound represented by Formula 1 below and the isomer of the compound into the first supply port 121 of the first distillation column 100; b) a step of discharging the introduced raw material to each of the first column top stream F₁₋₂ discharged from the upper section 110 of the first distillation column 100; and the first column top stream F₁₋₂, and the first, second and third column bottom streams F₁₋₃, F₁₋₄, and F₁₋₅ discharged from the lower section 130 of the first distillation column 100; c) a step of introducing the first column bottom stream F₁₋₃ into the second supply port 221 of the second distillation column 200; d) a step of discharging the stream introduced into the second supply port 221 to each of the second column top stream F₂₋₂ discharged from the upper section 210 of the second distillation column 200; and the fourth and fifth column bottom streams F₂₋₃ and F₂₋₄ discharged from the lower section 230 of the second distillation column 200; e) a step of exchanging heat between the second column top stream F₂₋₂ and the third column bottom stream F₁₋₅; and f) a step of separating the compound represented by Formula 1 from the upper section 210 of the second distillation column 200, and the isomer of the compound from the upper section 110 of the first distillation column 100:

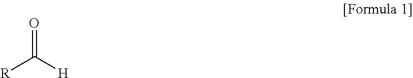

wherein R is a C₁ to C₁₂ an alkyl group.

The preparation method may be carried out using a distillation device with the aforementioned serial structure. The distillation device with the serial structure is the same as those described above, description thereof thus being omitted.

As described above, steps a) to f) are each independently, organically connected, and thus, boundaries therebetween are not clearly divided according to chronological order. Each of steps a) to f) may be carried out sequentially or independently at the same time.

The preparation method satisfies Equations 1 and 2 below. Descriptions thereof are the same as those described above, thus being omitted:

Tₜ₋₂−T_{b-3}≧8° C. [Equation 1]

P₂/P₁≧20 [Equation 2]

wherein Tₜ₋₂ indicates a temperature of the second column top stream and T_{b-3} indicates a temperature of the third column bottom stream F₁₋₅, and

P₁ indicates a pressure (kg/cm²g) of the upper section 110 of the first distillation column 100 and P₂ indicates a pressure (kg/cm²g) of the upper section 210 of the second distillation column 200.

### Advantageous Effects

A distillation device of the present application can minimize energy loss occurring during a purification process of a mixture including an isomer, for example, a raw material including n-butyl aldehyde and iso-butyl aldehyde, and can increase separation efficiency by obtaining a high-purity product.

### DESCRIPTION OF DRAWINGS

FIG. 1 exemplarily illustrates a distillation device according to an embodiment of the present application.

FIG. 2 exemplarily illustrates a distillation device according to another embodiment of the present application.

FIG. 3 exemplarily illustrates a general separation device used in a comparative example.

### BEST MODE

Now, the present invention will be described in more detail with reference to examples according to the present invention and comparative examples not according to the present invention. These examples are provided for illustrative purposes only and should not be construed as limiting the scope and spirit of the present invention.

### Example 1

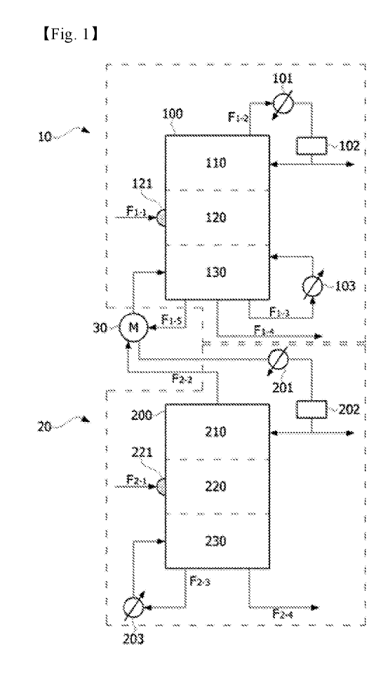

N-butyl aldehyde and iso-butyl aldehyde were separated by means of a distillation device illustrated in FIG. 1. In particular, a raw material including n-butyl aldehyde and iso-butyl aldehyde was introduced into each of a first distillation column with a theoretical plate number of 100 and a second distillation column with a theoretical plate number of 100. In this case, a ratio of the discharge rate of the raw material introduced into the first distillation column to the discharge rate of the raw material introduced into the second distillation column was controlled to 2:3.

A portion of a first column top stream discharged from an upper section of the first distillation column was refluxed in the upper section of the first distillation column via a first condenser. A portion of the remainder of the first column top stream was separated as a product including iso-butyl aldehyde and stored. A portion of a first column bottom stream discharged from a lower section of the first distillation column was refluxed in the lower section of the first distillation column via a first reboiler. A second column bottom stream discharged from the lower section of the first distillation column was separated as a product including n-butyl aldehyde and stored. A third column bottom stream discharged from the lower section of the first distillation column was introduced into a heat exchanger and heat-exchanged with a second column top stream of the second distillation column introduced into the heat exchanger, followed by being refluxed in the lower section of the first distillation column via the heat exchanger. In this case, operation pressure of the upper section of the first distillation column was adjusted to 0.02 Kg/cm²g and operation temperature thereof was adjusted to 65° C. Operation temperature of the lower section of the first distillation column was adjusted to 95° C.

Meanwhile, the second column top stream discharged from an upper section of the second distillation column was introduced into the heat exchanger and heat-exchanged with the third column bottom stream. Subsequently, a portion of the second column top stream having passed through the heat exchanger and a second condenser was refluxed in the upper section of the second distillation column, and a portion of the remainder of the second column top stream was separated as a product including iso-butyl aldehyde. A fourth column bottom stream discharged from a lower section of the second distillation column was refluxed in the lower section of the second distillation column via a second reboiler, and a fifth column bottom stream discharged from the lower section of the second distillation column was separated as a product including n-butyl aldehyde. In this case, operation pressure of the upper section of the second distillation column was adjusted to 2.5 Kg/cm²g, and operation temperature thereof was adjusted to 105° C. Operation temperature of the lower section of the second distillation column was adjusted to 129° C.

N-butyl aldehyde and iso-butyl aldehyde were separated by means of the distillation device of Example 1. With regard to this, used energy amount, recovery amount, reduction amount, reduction rate, and the purity of an n-butyl aldehyde/iso-butyl aldehyde product are summarized in Table 1 below.

### Example 2

N-butyl aldehyde and iso-butyl aldehyde were separated in the same manner as in Example 1, except that the operation conditions of the first and second distillation columns were changed as disclosed in Table 1 below.

N-butyl aldehyde and iso-butyl aldehyde were separated by means of a distillation device of Example 2. With regard to this, used energy amount, recovery amount, reduction amount, reduction rate, and the purity of an n-butyl aldehyde/iso-butyl aldehyde product are summarized in Table 1 below.

### Example 3

N-butyl aldehyde and iso-butyl aldehyde were separated in the same manner as in Example 1, except that the operation conditions of the first and second distillation columns were changed as disclosed in Table 1 below.

N-butyl aldehyde and iso-butyl aldehyde were separated by means of a distillation device of Example 3. With regard to this, used energy amount, recovery amount, reduction amount, reduction rate, and the purity of an n-butyl aldehyde/iso-butyl aldehyde product are summarized in Table 1 below.

### Example 4

N-butyl aldehyde and iso-butyl aldehyde were separated in the same manner as in Example 2, except that the operation conditions of the first and second distillation columns were changed as disclosed in Table 1 below.

N-butyl aldehyde and iso-butyl aldehyde were separated by means of a distillation device of Example 4. With regard to this, used energy amount, recovery amount, reduction amount, reduction rate, and the purity of an n-butyl aldehyde/iso-butyl aldehyde product are summarized in Table 1 below.

### Example 5

N-butyl aldehyde and iso-butyl aldehyde were separated in the same manner as in Example 2, except that the operation conditions of the first and second distillation columns were changed as disclosed in Table 1 below.

N-butyl aldehyde and iso-butyl aldehyde were separated by means of a distillation device of Example 5. With regard to this, used energy amount, recovery amount, reduction amount, reduction rate, and the purity of an n-butyl aldehyde/iso-butyl aldehyde product are summarized in Table 1 below.

### Comparative Example 1

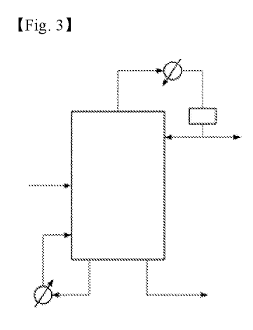

As illustrated in FIG. 3, n-butyl aldehyde and iso-butyl aldehyde were separated by means of one distillation column. A portion of a stream with a low boiling point discharged from an upper section of the distillation column was refluxed in the distillation column via a condenser, and a portion of the remainder of the stream was produced as a product including iso-butyl aldehyde. A portion of a stream discharged from a lower section of the distillation column was refluxed in the distillation column via a reboiler, and a portion of the remainder of the stream was separated as a product including n-butyl aldehyde. In this case, operation pressure of the upper section of the distillation column was adjusted to 0.32 Kg/cm²g, and the operation temperature thereof was adjusted to 73° C. Operation temperature of the lower section of the distillation column was adjusted to 100° C.

N-butyl aldehyde and iso-butyl aldehyde were separated by means of the distillation device of Comparative Example 1. With regard to this, used energy amount, recovery amount, reduction amount, reduction rate, and the purity of an n-butyl aldehyde/iso-butyl aldehyde product are summarized in Table 2 below.

### Comparative Example 2

N-butyl aldehyde and iso-butyl aldehyde were separated in the same manner as in Example 1, except that the operation conditions of the first and second distillation columns were changed as disclosed in Table 2 below.

N-butyl aldehyde and iso-butyl aldehyde were separated by means of a distillation device of Comparative Example 2. With regard to this, used energy amount, recovery amount, reduction amount, reduction rate, and the purity of an n-butyl aldehyde/iso-butyl aldehyde product are summarized in Table 2 below.

### Comparative Example 3

N-butyl aldehyde and iso-butyl aldehyde were separated in the same manner as in Example 1, except that the operation conditions of the first and second distillation columns were changed as disclosed in Table 2 below.

N-butyl aldehyde and iso-butyl aldehyde were separated by means of a distillation device of Comparative Example 3. With regard to this, used energy amount, recovery amount, reduction amount, reduction rate, and the purity of an n-butyl aldehyde/iso-butyl aldehyde product are summarized in Table 2 below.

### Comparative Example 4

N-butyl aldehyde and iso-butyl aldehyde were separated in the same manner as in Example 1, except that the operation conditions of the first and second distillation columns were changed as disclosed in Table 2 below.

N-butyl aldehyde and iso-butyl aldehyde were separated by means of a distillation device of Comparative Example 4. With regard to this, used energy amount, recovery amount, reduction amount, reduction rate, and the purity of an n-butyl aldehyde/iso-butyl aldehyde product are summarized in Table 2 below.

### Comparative Example 5

N-butyl aldehyde and iso-butyl aldehyde were separated in the same manner as in Example 1, except that the operation conditions of the first and second distillation columns were changed as disclosed in Table 3 below.

N-butyl aldehyde and iso-butyl aldehyde were separated by means of a distillation device of Comparative Example 5. With regard to this, used energy amount, recovery amount, reduction amount, reduction rate, and the purity of an n-butyl aldehyde/iso-butyl aldehyde product are summarized in Table 3 below.

### Comparative Example 6

N-butyl aldehyde and iso-butyl aldehyde were separated in the same manner as in Example 1, except that the operation conditions of the first and second distillation columns were changed as disclosed in Table 3 below.

N-butyl aldehyde and iso-butyl aldehyde were separated by means of a distillation device of Comparative Example 6. With regard to this, used energy amount, recovery amount, reduction amount, reduction rate, and the purity of an n-butyl aldehyde/iso-butyl aldehyde product are summarized in Table 3 below.

### Comparative Example 7

N-butyl aldehyde and iso-butyl aldehyde were separated in the same manner as in Example 1, except that the operation conditions of the first and second distillation columns were changed as disclosed in Table 3 below.

N-butyl aldehyde and iso-butyl aldehyde were separated by means of a distillation device of Comparative Example 7. With regard to this, used energy amount, recovery amount, reduction amount, reduction rate, and the purity of an n-butyl aldehyde/iso-butyl aldehyde product are summarized in Table 3 below.

### Comparative Example 8

N-butyl aldehyde and iso-butyl aldehyde were separated in the same manner as in Example 1, except that the operation conditions of the first and second distillation columns were changed as disclosed in Table 3 below.

N-butyl aldehyde and iso-butyl aldehyde were separated by means of a distillation device of Comparative Example 8. With regard to this, used energy amount, recovery amount, reduction amount, reduction rate, and the purity of an n-butyl aldehyde/iso-butyl aldehyde product are summarized in Table 3 below.

TABLE 1

| | | Example 1 | Example 2 | Example 3 | Example 4 | Example 5 |
|---|---|---|---|---|---|---|
| Discharge | First | 40 | 50 | 60 | 50 | 50 |
| rate of raw | distillation | | | | | |
| material (%) | column | | | | | |
| | Second | 60 | 50 | 40 | 50 | 50 |
| | distillation | | | | | |
| | column | | | | | |
| Pressure of | First | 0.02 | 0.02 | 0.02 | 0.07 | 0.1 |
| upper section | distillation | | | | | |
| (kg/cm²g) | column | | | | | |
| | Second | 2.5 | 2.5 | 2.5 | 2.5 | 2.5 |
| | distillation | | | | | |
| | column | | | | | |
| Column | First | 65/95 | 65/95 | 65/95 | 66/96 | 67/97 |
| temperature | distillation | | | | | |
| (°C.) (upper | column | | | | | |
| section/lower | Second | 105/129 | 105/129 | 105/129 | 105/129 | 105/129 |
| section) | distillation | | | | | |
| | column | | | | | |
| Energy | First | 4.69 | 5.49 | 6.59 | 5.65 | 5.87 |
| (Gcal/hr) | distillation | | | | | |
| | column | | | | | |
| | Second | 7.99 | 7.7 | 7.15 | 7.7 | 7.7 |
| | distillation | | | | | |
| | column | | | | | |
| | Recovery | 4.69 | 5.49 | 4.95 | 5.04 | 5.04 |
| | amount | | | | | |
| | Total | 7.99 | 7.7 | 8.79 | 8.31 | 8.53 |
| | Reduction | 4.04 | 4.33 | 3.24 | 3.72 | 3.5 |
| | amount | | | | | |
| | Energy | | | | | |
| | reduction | 33.6 | 36.0 | 26.9 | 30.9 | 29.1 |
| | rate (%) | | | | | |

| | | | | | |
|---|---|---|---|---|---|
| Product purity (n-BAL/iso- | 99.7/99.0 | 99.7/99.0 | 99.7/99.0 | 99.7/99.0 | 99.7/99.0 |
| BAL) | | | | | |

TABLE 2

| | | Comparative | Comparative | Comparative | Comparative |
|---|---|---|---|---|---|
| | | Example 1 | Example 2 | Example 3 | Example 4 |
| Discharge rate | First | — | 50 | 60 | 40 |
| of raw | distillation | | | | |
| material (%) | column | | | | |
| | Second | 100 | 50 | 40 | 60 |
| | distillation | | | | |
| | column | | | | |
| Pressure of | First | — | 0.13 | 0.13 | 0.13 |
| upper section | distillation | | | | |
| (kg/cm²g) | column | | | | |
| | Second | 0.32 | 2.2 | 2.2 | 2.2 |
| | distillation | | | | |
| | column | | | | |
| Column | First | — | 68/97 | 68/97 | 68/97 |
| temperature | distillation | | | | |
| (° C.) (upper | column | | | | |
| section/lower | Second | 73/100 | 104/127 | 104/127 | 104/127 |
| section) | distillation | | | | |
| | column | | | | |
| Energy | First | — | 6.37 | 5.84 | 5.46 |
| (Gcal/hr) | distillation | | | | |
| | column | | | | |
| | Second | 12.03 | 7.41 | 8.38 | 8.76 |
| | distillation | | | | |
| | column | | | | |
| | Recovery | — | 4.91 | 4.88 | 5.06 |
| | amount | | | | |
| | Total | 12.03 | 8.87 | 9.34 | 9.16 |
| | Reduction | — | 3.16 | 2.69 | 2.87 |
| | amount | | | | |
| | Energy | — | 26.3 | 22.4 | 23.9 |
| | reduction | | | | |
| | rate (%) | | | | |

| | | | | |
|---|---|---|---|---|
| Product purity (n-BAL/iso-BAL) | 99.7/99.0 | 99.7/99.0 | 99.7/99.0 | 99.7/99.0 |

TABLE 3

| | | Comparative | Comparative | Comparative | Comparative |
|---|---|---|---|---|---|
| | | Example 5 | Example 6 | Example 7 | Example 8 |
| Discharge rate | First | 20 | 80 | 50 | 50 |
| of raw | distillation | | | | |
| material (%) | column | | | | |
| | Second | 80 | 20 | 50 | 50 |
| | distillation | | | | |
| | column | | | | |
| Pressure of | First | 0.02 | 0.02 | 0.17 | 0.1 |
| upper section | distillation | | | | |
| (kg/cm²g) | column | | | | |
| | Second | 2.5 | 2.5 | 2.5 | 2.2 |
| | distillation | | | | |
| | column | | | | |
| Column | First | 65/95 | 65/95 | 69/97 | 67/96 |
| temperature | distillation | | | | |
| (° C.) (upper | column | | | | |
| section/lower | Second | 105/129 | 105/129 | 105/129 | 102/125 |
| section) | distillation | | | | |
| | column | | | | |
| Energy | First | 2.20 | 8.79 | 7.07 | 7.02 |
| (Gcal/hr) | distillation | | | | |
| | column | | | | |
| | Second | 14.31 | 3.58 | 7.70 | 7.65 |
| | distillation | | | | |
| | column | | | | |
| | Recovery | 2.20 | 2.21 | 4.72 | 4.99 |
| | amount | | | | |
| | Total | 14.20 | 10.16 | 10.05 | 9.31 |
| | Reduction | −2.17 | 1.87 | 1.98 | 2.35 |
| | amount | | | | |
| | Energy | — | 15.5 | 16.5 | 19.5 |
| | reduction | | | | |
| | rate(%) | | | | |

| | | | | |
|---|---|---|---|---|
| Product purity (n-BAL/iso-BAL) | 99.7/99.0 | 99.7/99.0 | 99.7/99.0 | 99.7/99.0 |

As shown in Tables 1 to 3, it can be confirmed that, when the isomer of n-butyl aldehyde is separated according to each of Examples 1 to 5, a total energy consumption amount is greatly decreased, compared to the comparative examples. Accordingly, it can be confirmed that, when the raw material was separated by means of the distillation device of each of Examples 1 to 5 of the present application, an energy reduction effect of up to 36.0% is achieved, compared to the case in which the distillation device of Comparative Example 1 is used.

In addition, it can be confirmed that, as shown in the examples and the comparative examples, n-butyl aldehyde and iso-butyl aldehyde may be separated in high purity and efficiency by controlling a temperature difference between the lower section of the first distillation column and the upper section of the second distillation column and the pressures of the upper sections of the first and second distillation columns within a specific range.

In addition, as shown in Comparative Examples 5 and 6, it can be confirmed that n-butyl aldehyde and iso-butyl aldehyde may be separated in high purity and efficiency by controlling a ratio of the discharge rate into each of the first and second distillation columns within a specific range.

### Example 6

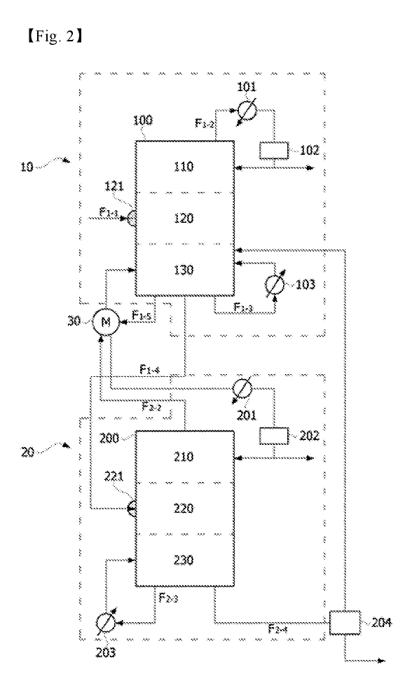

N-butyl aldehyde and iso-butyl aldehyde were separated by means of a distillation device illustrated in FIG. 2. In particular, a raw material including n-butyl aldehyde and iso-butyl aldehyde was introduced into a first distillation column with a theoretical plate number of 100.

A portion of a first column top stream discharged from an upper section of the first distillation column was refluxed in the upper section of the first distillation column via a first condenser. A portion of the remainder of the first column top stream was separated as a product including iso-butyl aldehyde and stored. A portion of a first column bottom stream discharged from a lower section of the first distillation column was refluxed in the lower section of the first distillation column via a first reboiler. A second column bottom stream discharged from the lower section of the first distillation column was introduced into a second distillation column. A third column bottom stream discharged from the lower section of the first distillation column was introduced into a heat exchanger and heat-exchanged with a second column top stream of the second distillation column introduced into the heat exchanger, followed by being refluxed in the lower section of the first distillation column via the heat exchanger. In this case, operation pressure of the upper section of the first distillation column was adjusted to 0.07 Kg/cm²g and operation temperature thereof was adjusted to 65° C. Operation temperature of the lower section of the first distillation column was adjusted to 96° C.

Meanwhile, the second column top stream discharged from an upper section of the second distillation column was introduced into the heat exchanger and heat-exchanged with the third column bottom stream. Subsequently, a portion of the second column top stream having passed through the heat exchanger and a second condenser was refluxed in the upper section of the second distillation column, and a portion of the remainder of the second column top stream was separated as a product including n-butyl aldehyde. In this case, the purity of n-butyl aldehyde was 99.9%. A fourth column bottom stream discharged from a lower section of the second distillation column was refluxed in the lower section of the second distillation column via a second reboiler, and a fifth column bottom stream discharged from the lower section of the second distillation column was separated as a product including n-butyl aldehyde. In this case, operation pressure of the upper section of the second distillation column was adjusted to 1.4 Kg/cm²g, and operation temperature thereof was adjusted to 105° C. Operation temperature of the lower section of the second distillation column was adjusted to 120° C.

N-butyl aldehyde and iso-butyl aldehyde were separated by means of the distillation device of Example 6. With regard to this, used energy amount, recovery amount, reduction amount, reduction rate, and the purity of an n-butyl aldehyde/iso-butyl aldehyde product are summarized in Table 4 below.

### Example 7

N-butyl aldehyde and iso-butyl aldehyde were separated in the same manner as in Example 6, except that the operation conditions of the first and second distillation columns were changed as disclosed in Table 3 below.

N-butyl aldehyde and iso-butyl aldehyde were separated by means of a distillation device of Example 7. With regard to this, used energy amount, recovery amount, reduction amount, reduction rate, and the purity of an n-butyl aldehyde/iso-butyl aldehyde product are summarized in Table 4 below.

### Example 8

N-butyl aldehyde and iso-butyl aldehyde were separated in the same manner as in Example 6, except that the operation conditions of the first and second distillation columns were changed as disclosed in Table 3 below.

N-butyl aldehyde and iso-butyl aldehyde were separated by means of a distillation device of Example 8. With regard to this, used energy amount, recovery amount, reduction amount, reduction rate, and the purity of an n-butyl aldehyde/iso-butyl aldehyde product are summarized in Table 4 below.

### Comparative Example 9

N-butyl aldehyde and iso-butyl aldehyde were separated in the same manner as in Example 6, except that the operation conditions of the first and second distillation columns were changed as disclosed in Table 5 below.

N-butyl aldehyde and iso-butyl aldehyde were separated by means of a distillation device of Comparative Example 9. With regard to this, used energy amount, recovery amount, reduction amount, reduction rate, and the purity of an n-butyl aldehyde/iso-butyl aldehyde product are summarized in Table 5 below.

### Comparative Example 10

N-butyl aldehyde and iso-butyl aldehyde were separated in the same manner as in Example 6, except that the operation conditions of the first and second distillation columns were changed as disclosed in Table 5 below.

N-butyl aldehyde and iso-butyl aldehyde were separated by means of a distillation device of Comparative Example 10. With regard to this, used energy amount, recovery amount, reduction amount, reduction rate, and the purity of an n-butyl aldehyde/iso-butyl aldehyde product are summarized in Table 5 below.

### Comparative Example 11

N-butyl aldehyde and iso-butyl aldehyde were separated in the same manner as in Example 6, except that the operation conditions of the first and second distillation columns were changed as disclosed in Table 5 below.

N-butyl aldehyde and iso-butyl aldehyde were separated by means of a distillation device of Comparative Example 11. With regard to this, used energy amount, recovery amount, reduction amount, reduction rate, and the purity of an n-butyl aldehyde/iso-butyl aldehyde product are summarized in Table 5 below.

### Comparative Example 12

N-butyl aldehyde and iso-butyl aldehyde were separated in the same manner as in Example 6, except that the operation conditions of the first and second distillation columns were changed as disclosed in Table 6 below.

N-butyl aldehyde and iso-butyl aldehyde were separated by means of a distillation device of Comparative Example 12. With regard to this, used energy amount, recovery amount, reduction amount, reduction rate, and the purity of an n-butyl aldehyde/iso-butyl aldehyde product are summarized in Table 6 below.

### Comparative Example 13

N-butyl aldehyde and iso-butyl aldehyde were separated in the same manner as in Example 6, except that the operation conditions of the first and second distillation columns were changed as disclosed in Table 6 below.

N-butyl aldehyde and iso-butyl aldehyde were separated by means of a distillation device of Comparative Example 13. With regard to this, used energy amount, recovery amount, reduction amount, reduction rate, and the purity of an n-butyl aldehyde/iso-butyl aldehyde product are summarized in Table 6 below.

TABLE 4

| | | Example 6 | Example 7 | Example 8 |
|---|---|---|---|---|
| Pressure of | First | 0.07 | 0.07 | 0.1 |
| upper section | distillation | | | |
| (kg/cm²g) | column | | | |
| | Second | 1.4 | 1.8 | 2.1 |
| | distillation | | | |
| | column | | | |
| Column | First | 66/96 | 66/96 | 67/97 |
| temperature | distillation | | | |
| (° C.) (upper | column | | | |
| section/lower | Second | 105/120 | 110/124 | 114/128 |
| section) | distillation | | | |
| | column | | | |
| Energy | First | 10.91 | 10.91 | 11.45 |
| (Gcal/hr) | distillation | | | |
| | column | | | |
| | Second | 5.71 | 5.82 | 6.01 |
| | distillation | | | |
| | column | | | |
| | Recovery | 4.59 | 4.72 | 4.80 |
| | amount | | | |
| | Total | 12.03 | 12.01 | 12.66 |
| | Reduction | 2.34 | 2.36 | 1.71 |
| | amount | | | |
| | Energy | 16.3 | 16.4 | 11.9 |
| | reduction | | | |
| | rate (%) | | | |

| | | | |
|---|---|---|---|
| Product purity (n-BAL/iso-BAL) | 99.9/99.3 | 99.9/99.3 | 99.9/99.3 |

TABLE 5

| | | Comparative | Comparative | Comparative |
|---|---|---|---|---|
| | | Example 9 | Example 10 | Example 11 |
| Pressure of | First | 0.34 | 0.18 | 0.052 |
| upper section | distillation | | | |
| (kg/cm²g) | column | | | |
| | Second | 0.15 | 1.4 | 1.2 |
| | distillation | | | |
| | column | | | |
| Column | First | 73/100 | 69/98 | 66/95 |
| temperature | distillation | | | |
| (° C.) (upper | column | | | |
| section/lower | Second | 79/97 | 105/120 | 101/116 |
| section) | distillation | | | |
| | column | | | |
| Energy | First | 12.07 | 11.79 | 10.91 |
| (Gcal/hr) | distillation | | | |
| | column | | | |
| | Second | 2.3 | 5.67 | 5.65 |
| | distillation | | | |
| | column | | | |
| | Recovery | — | 3.94 | 3.43 |
| | amount | | | |
| | Total | 14.37 | 13.52 | 13.13 |
| | Reduction | — | 0.85 | 1.24 |
| | amount | | | |
| | Energy | — | 5.9 | 8.6 |
| | reduction | | | |
| | rate (%) | | | |

| | | | |
|---|---|---|---|
| Product purity (n-BAL/iso-BAL) | 99.9/99.3 | 99.9/99.3 | 99.9/99.3 |

TABLE 6

| | | Comparative | Comparative |
|---|---|---|---|
| | | Example 12 | Example 13 |
| Pressure of | First | 0.11 | 0.3 |
| upper section | distillation | | |
| (kg/cm²g) | column | | |
| | Second | 1.35 | 1.6 |
| | distillation | | |
| | column | | |
| Column temperature | First | 67/96 | 72/100 |
| (° C.) (upper | distillation | | |
| section/lower | column | | |
| section) | Second | 104/167 | 108/170 |
| | distillation | | |
| | column | | |
| Energy | First | 11.5 | 11.9 |
| (Gcal/hr) | distillation | | |
| | column | | |
| | Second | 5.60 | 6.32 |
| | distillation | | |
| | column | | |
| | Recovery | 4.38 | 3.88 |
| | amount | | |
| | Total | 12.72 | 14.34 |
| | Reduction | 1.65 | 0.03 |
| | amount | | |
| | Energy | 11.5 | 0.2 |
| | reduction | | |
| | rate (%) | | |

| | | |
|---|---|---|
| Product purity (n-BAL/iso-BAL) | 99.9/99.3 | 99.9/99.3 |

As shown in Tables 4 to 6, it can be confirmed that, when the isomer of n-butyl aldehyde is separated according to each of Examples 6 to 8, a total energy consumption amount is greatly decreased, compared to the comparative examples. Accordingly, it can be confirmed that, when the raw material was separated by means of the distillation device of each of Examples 6 to 8 of the present application, an energy reduction effect of up to 16.4% is achieved, compared to the case in which the distillation device of Comparative Example 5 is used.

In addition, it can be confirmed that, as shown in the examples and the comparative examples, n-butyl aldehyde may be separated in high purity and efficiency by controlling a temperature difference between the lower section of the first distillation column and the upper section of the second distillation column and the pressures of the upper sections of the first and second distillation columns, within a specific range.

1. A distillation device, comprising a first distillation unit that comprises a first condenser, a first reboiler and a first distillation column; a second distillation unit that comprises a second condenser, a second reboiler, and a second distillation column; and a heat exchanger,
wherein a raw material comprising a compound represented by Formula 1 below and an isomer of the compound is introduced into a first supply port of the first distillation column and/or a second supply port of the second distillation column,
the raw material introduced into the first supply port of the first distillation column is separately discharged into each of a first column top stream discharged from an upper section of the first distillation column; and first, second and third column bottom streams separately discharged from a lower section of the first distillation column,
the first column top stream is introduced into the first condenser, and a portion or all of the first column top stream via the first condenser is refluxed in the upper section of the first distillation column,
the first column bottom stream is introduced into the first reboiler, and the first column bottom stream via the first reboiler is refluxed in the lower section of the first distillation column,
the stream introduced into the second supply port of the second distillation column is separately discharged to each of a second column top stream discharged from an upper section of the second distillation column; and fourth and fifth column bottom streams discharged from a lower section of the second distillation column,
the fourth column bottom stream is introduced into the second reboiler, and the fourth column bottom stream via the second reboiler is refluxed in the lower section of the second distillation column,
the third column bottom stream and the second column top stream are introduced into the heat exchanger and exchange heat therebetween, the third column bottom stream via the heat exchanger is refluxed in the lower section of the first distillation column, the second column top stream via the heat exchanger is introduced into the second condenser, and a portion or all of the second column top stream via the second condenser is refluxed in the upper section of the second distillation column, and
Equations 1 and 2 are satisfied: 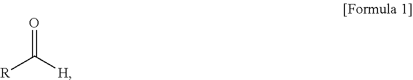
wherein R is a C₁ to C₁₂ alkyl group;
Tₜ₋₂−T_{b-3}≧8° C. [Equation 1]
P₂/P₁≧20 [Equation 2]
wherein Tₜ₋₂ indicates a temperature of the second column top stream, and T_{b-3} indicates a temperature of the third column bottom stream, and
P₁ indicates a pressure of the upper section of the first distillation column (kg/cm²g), and P₂ indicates a pressure of the upper section of the second distillation column (kg/cm²g).

2. The distillation device according to claim 1, wherein the raw material is introduced into each of the first supply port of the first distillation column and the second supply port of the second distillation column.

3. The distillation device according to claim 2, wherein the compound represented by Formula 1 is n-butyl aldehyde, and the isomer of the compound is iso-butyl aldehyde,
wherein a content of the iso-butyl aldehyde in each of the first column top stream and the second column top stream is 90% or more, and a content of the n-butyl aldehyde in each of the second column bottom stream and the fifth column bottom stream is 90% or more.

4. The distillation device according to claim 2, wherein the distillation device satisfies Equation 3 below:
0.3≦F₁/F₂≦3.0, [Equation 3]
wherein F₁ indicates a discharge rate of the raw material (ton/hr) introduced into the first supply port of the first distillation column, and F₂ indicates a discharge rate of the raw material (ton/hr) introduced into the second supply port of the second distillation column.

5. The distillation device according to claim 1, wherein a pressure of the upper section of the first distillation column is 0.01 to 0.1 kg/cm²g.

6. The distillation device according to claim 1, wherein a pressure of the upper section of the second distillation column is 2.3 to 2.7 kg/cm²g.

7. The distillation device according to claim 1, wherein a temperature of the upper section of the first distillation column is 60 to 70° C.

8. The distillation device according to claim 1, wherein a temperature of the lower section of the first distillation column is 90 to 100° C.

9. The distillation device according to claim 1, wherein a temperature of the upper section of the second distillation column is 100 to 110° C.

10. The distillation device according to claim 1, wherein a temperature of the lower section of the second distillation column is 120 to 140° C.

11. The distillation device according to claim 1, wherein the raw material is supplied to the first supply port of the first distillation column, and the second column bottom stream of the first distillation column is supplied to the second supply port of the second distillation column.

12. The distillation device according to claim 11, wherein the compound represented by Formula 1 is n-butyl aldehyde, and the isomer of the compound is iso-butyl aldehyde,
wherein a content of the iso-butyl aldehyde in the first column top stream is 90% or more, and a content of the n-butyl aldehyde in the second column top stream is 90% or more.

13. The distillation device according to claim 11, wherein a portion of the fifth column bottom stream discharged from the lower section of the second distillation column is introduced into the lower section of the first distillation column.

14. The distillation device according to claim 11, wherein a pressure of the upper section of the first distillation column is 0.01 to 0.1 kg/cm²g.

15. The distillation device according to claim 11, wherein a pressure of the upper section of the second distillation column is 1.0 to 2.0 kg/cm²g.

16. The distillation device according to claim 11, wherein a temperature of the upper section of the first distillation column is 60 to 70° C.

17. The distillation device according to claim 11, wherein a temperature of the lower section of the first distillation column is 90 to 100° C.

18. The distillation device according to claim 11, wherein a temperature of the upper section of the second distillation column is 100 to 110° C.

19. The distillation device according to claim 11, wherein a temperature of the lower section of the second distillation column is 120 to 140° C.

20. A method of preparing a compound represented by Formula 1, the method comprising: introducing a raw material comprising the compound represented by Formula 1 below and an isomer of the compound into each of a first supply port of a first distillation column and a second supply port of a second distillation column;
discharging the raw material introduced into the first supply port to each of a first column top stream discharged from an upper section of the first distillation column; and first, second and third column bottom streams discharged from a lower section of the first distillation column;
discharging the raw material introduced into the second supply port to each of a second column top stream discharged from an upper section of the second distillation column; and fourth and fifth column bottom streams discharged from a lower section of the second distillation column;
heat-exchanging the second column top stream with the third column bottom stream; and
separating the compound represented by Formula 1 from the lower section of the first distillation column, and the isomer of the compound represented by Formula 1 from the upper section of the first distillation column and the upper section of the second distillation column,
wherein Equations 1 and 2 below are satisfied: 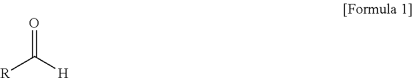
wherein R is a C₁ to C₁₂ alkyl group;
Tₜ₋₂−T_{b-3}≧8° C. [Equation 1]
P₂/P₁≧20 [Equation 2]
wherein Tₜ₋₂ indicates a temperature of the second column top stream, and T_{b-3} indicates a temperature of the third column bottom stream, and
P₁ indicates a pressure of the upper section of the first distillation column (kg/cm²g), and P₂ indicates a pressure of the upper section of the second distillation column (kg/cm²g).

21. A method of preparing a compound represented by Formula 1, the method comprising: introducing a raw material comprising the compound represented by Formula 1 below and an isomer of the compound into a first supply port of a first distillation column;
discharging the introduced raw material to each of a first column top stream discharged from an upper section of the first distillation column; and first, second and third column bottom streams discharged from a lower section of the first distillation column;
introducing the first column bottom stream into a second supply port of a second distillation column,
discharging the stream introduced into the second supply port to each of a second column top stream discharged from an upper section of the second distillation column; and fourth and fifth column bottom streams discharged from a lower section of the second distillation column;
heat-exchanging the second column top stream with the third column bottom stream; and
separating the compound represented by Formula 1 from the upper section of the second distillation column, and the isomer of the compound represented by Formula 1 from the upper section of the first distillation column,
wherein Equations 1 and 2 below are satisfied: 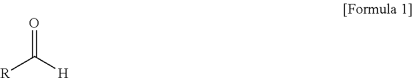
wherein R is a C₁ to C₁₂ alkyl group;
Tₜ₋₂−T_{b-3}≧8° C. [Equation 1]
P₂/P₁≧20 [Equation 2]
wherein Tₜ₋₂ indicates a temperature of the second column top stream, and T_{b-3} indicates a temperature of the third column bottom stream, and
P₁ indicates a pressure of the upper section of the first distillation column (kg/cm²g), and P₂ indicates a pressure of the upper section of the second distillation column (kg/cm²g).
